(19) United States
(12) Patent Application Publication
(10) Pub. No.: US 2010/0191341 A1
(43) Pub. Date: Jul. 29, 2010

(54) LATERAL CONDYLE POSTERIOR INFLECTION FOR TOTAL KNEE IMPLANT

(75) Inventor: Christopher M. Byrd, Elkhart, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 12/694,771

(22) Filed: Jan. 27, 2010

Related U.S. Application Data
(60) Provisional application No. 61/147,801, filed on Jan. 28, 2009.

(51) Int. Cl. A61F 2/38

(52) U.S. Cl. 623/20.3; 623/20.21; 623/20.31

(57) ABSTRACT

A prosthetic knee includes a femoral component and a tibial component that cooperate to promote joint stability in deep flexion of the knee. An articulating surface of the femoral component transitions from a convex curvature to a concave curvature at a femoral inflection point. An articulating surface of the tibial component transitions from a concave curvature to a convex curvature at a tibial inflection point. The femoral and tibial inflection points cooperate during flexion of the knee so that the concave curvature of the femoral component mates with the convex curvature of the tibial component.

### CROSS REFERENCE TO RELATED APPLICATION

This application claims the benefit under Title 35, U.S.C. §119(e) of U.S. Provisional Patent Application Ser. No. 61/147,801, entitled LATERAL CONDYLE POSTERIOR INFLECTION FOR TOTAL KNEE IMPLANT, filed on Jan. 28, 2009, the entire disclosure of which is expressly incorporated herein by reference.

### BACKGROUND

1. Technical Field

The present disclosure relates to orthopedic prostheses. More particularly, the present disclosure relates to knee prostheses.

2. Description of the Related Art

In a natural knee joint, flexion-extension involves various complex movements between the femur and the tibia. The femur does not merely pivot about a transverse axis relative to the tibia like a hinge joint, but also has other rotational and translational movement relative to the tibia. For example, in addition to a pivot motion, the knee joint undergoes both translational movement and rotational movement about a sagittal axis. During flexion and extension of the knee, the femur may translate anteriorly-posteriorly across the tibia, and/or the tibia may rotate internally-externally about its longitudinal axis relative to the femur.

Disease and trauma affecting the articular surfaces of a knee joint are commonly treated by surgically replacing the articulating ends of the femur and tibia with prosthetic femoral and tibial components. On one hand, the prosthetic knee should be designed to maximize the range of motion between the femoral and tibial components and to simulate the complex movements of the natural knee joint. On the other hand, articulation between the femoral and tibial components should be constrained to prevent dislocation.

### SUMMARY

The present disclosure provides a knee prosthesis including a femoral component and a tibial component that cooperate to facilitate joint stability in deep flexion of the knee. An articulating surface of the femoral component transitions from a convex curvature to a concave curvature at a femoral inflection point. An articulating surface of the tibial component transitions from a concave curvature to a convex curvature at a tibial inflection point. The femoral and tibial inflection points cooperate during deep flexion of the knee joint so that the concave curvature of the femoral component mates with the convex curvature of the tibial component.

In one form thereof, the present invention provides a prosthetic knee including a femoral component and a tibial component. The femoral component has an anterior femoral end and a posterior femoral end, and is configured for securement to a resected distal femur. The femoral component includes a femoral articulating surface extending in a sagittal plane between the anterior femoral end and the posterior femoral end, the femoral articulating surface having a femoral inflection point in the sagittal plane. The femoral articulating surface transitions in the sagittal plane from a convex curvature to a concave curvature at the femoral inflection point. The tibial component has an anterior tibial end and a posterior tibial end, and is configured for securement to a resected proximal tibia. The tibial component includes a tibial articulating surface extending between the anterior tibial end and the posterior tibial end, with the tibial articulating surface configured to articulate with the femoral articulating surface. The tibial articulating surface contacts the femoral inflection point at an angle of flexion of the prosthetic knee.

In one aspect, the angle of flexion of the prosthetic knee at which the tibial articulating surface contacts the femoral inflection point equals at least 130 degrees of flexion of the prosthetic knee.

In another form thereof, the present invention provides a prosthetic knee including a femoral component and a tibial component. The femoral component has an anterior femoral end and a posterior femoral end, the femoral component is configured for securement to a resected distal femur. The femoral component includes a medial condyle, a lateral condyle, and a femoral inflection point. The medial condyle includes a medial femoral articulating surface extending between the anterior femoral end and the posterior femoral end, the medial femoral articulating surface having a convex medial condyle curvature. The lateral condyle includes a lateral femoral articulating surface extending between the anterior femoral end and the posterior femoral end, the lateral femoral articulating surface having a substantially convex lateral condyle curvature. The femoral inflection point is disposed on the lateral femoral articulating surface adjacent the posterior femoral end, the lateral femoral articulating surface transitioning from the substantially convex lateral condyle curvature to a concave lateral condyle curvature at the femoral inflection point, and the concave lateral condyle curvature having a concave femoral radius. The tibial component has an anterior tibial end and a posterior tibial end, the tibial component configured for securement to a resected proximal tibia. The tibial component includes a medial tibial compartment, a lateral tibial compartment, and a tibial inflection point. The medial tibial compartment has a medial articulating surface extending between the anterior tibial end and the posterior tibial end, the medial tibial articulating surface sized and positioned to articulate with the medial femoral articulating surface of the medial condyle. The lateral tibial compartment has a lateral articulating surface extending between the anterior tibial end and the posterior tibial end, the lateral tibial articulating surface sized and positioned to articulate with the lateral femoral articulating surface of the lateral condyle. The tibial inflection point is disposed on the lateral tibial articulating surface adjacent the posterior tibial end, the lateral tibial articulating surface transitioning from a concave tibial curvature to a convex tibial curvature at the tibial inflection point, the convex tibial curvature defining a convex tibial radius, the concave femoral radius at least as great as the convex tibial radius.

In yet another aspect thereof, the present invention provides a method of stabilizing a prosthetic knee in a configuration corresponding to deep flexion of a knee. The method includes: providing a femoral component having a femoral articulating surface with a convex femoral curvature and a concave femoral curvature, the convex femoral curvature transitioning to the concave femoral curvature at a femoral inflection point; providing a tibial component having a tibial articulating surface with a concave tibial curvature and a convex tibial curvature, the convex tibial curvature transitioning to the concave tibial curvature at a tibial inflection point; and articulating the femoral component with respect to the tibial component from an extension orientation to a flexion orientation, the convex femoral curvature engaging the concave tibial curvature in the extension orientation, and the concave femoral curvature engaging the convex tibial curvature in the flexion orientation.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is an exploded lateral view of a femoral component and a tibial component of an exemplary knee arthroplasty system;

FIG. 2 is a lateral view of the knee arthroplasty system of FIG. 1 in an extended position; and

FIG. 3 is a lateral view of the knee arthroplasty system of FIG. 1 in deep flexion.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

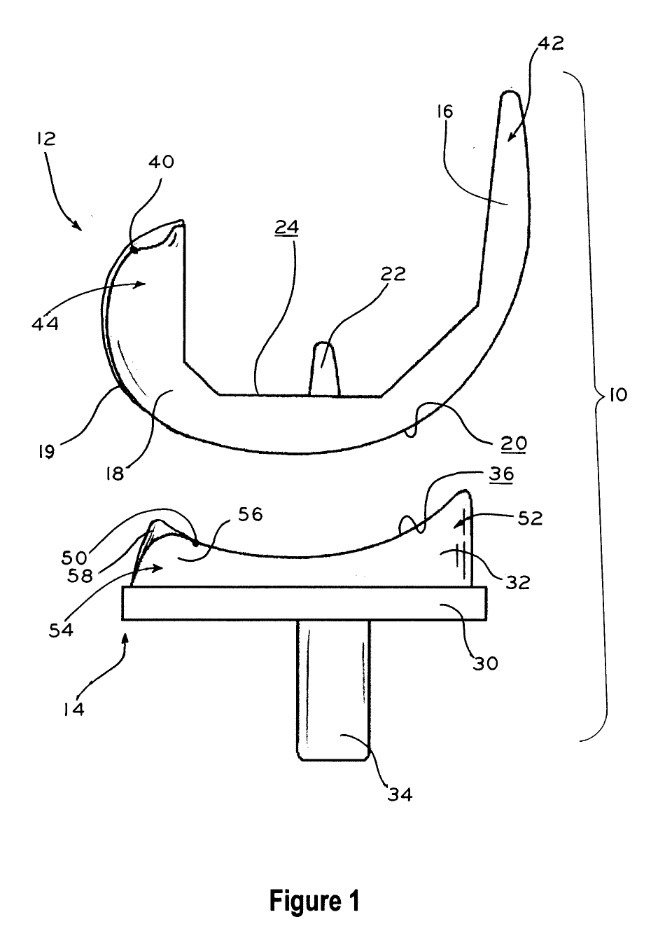

Referring to FIG. 1, the lateral side of total knee arthroplasty system or prosthetic knee 10, which is configured for use in a patient's right knee, is illustrated. However, the principles of the present disclosure are also applicable to a left knee. Knee arthroplasty system 10 includes femoral component 12 and tibial component 14 which each have convex/concave inflection points that cooperate to provide a high degree of joint stability in deep flexion, as described in detail below.

Femoral component 12 is configured for securement to a resected distal femur (not shown). Femoral component 12 includes anterior flange 16 that is configured to articulate with a natural or prosthetic patella (not shown). Femoral component 12 also includes lateral condyle 18 and an opposing medial condyle 19 that extend from anterior flange 16. Anterior flange 16, lateral condyle 18, and medial condyle 19, cooperate to define a substantially convex femoral articulating surface 20. Femoral component 12 may also include at least one fixation mechanism, such as peg 22. With femoral component 12 resting against the resected distal femur, peg 22 extends proximally into the distal femur. Femoral component 12 may be constructed of a biocompatible ceramic or metal, including, but not limited to, titanium, a titanium alloy, cobalt chromium, or cobalt chromium molybdenum, for example.

Tibial component 14 is configured for securement to a resected proximal tibia (not shown). Tibial component 14 includes base 30 and bearing portion 32. Base 30 may include at least one fixation mechanism, such as stem 34. With base 30 of tibial component 14 resting atop the resected proximal tibia, stem 34 extends distally into the proximal tibia. Base 30 of tibial component 14 may be constructed of a biocompatible ceramic or metal, including, but not limited to, titanium, a titanium alloy, cobalt chromium, cobalt chromium molybdenum, porous tantalum, or a highly porous biomaterial, for example. An exemplary highly porous biomaterial is produced using Trabecular Metal® technology generally available from Zimmer, Inc., of Warsaw, Ind. Trabecular Metal® is a trademark of Zimmer, Inc., of Warsaw, Ind. A highly porous biomaterial may be useful as a bone substitute and as cell and tissue receptive material.

Referring still to FIG. 1, bearing portion 32 of tibial component 14 is positioned atop base 30. Bearing portion 32 may be fixedly attached to base 30, such as by a snap-fit or with adhesive, or bearing portion 32 may be moveable relative to base 30 to form a mobile bearing component. Bearing portion 32 includes lateral compartment 56 and medial compartment 58. Lateral compartment 56 and medial compartment 58 of bearing portion 32 cooperate to define a substantially concave tibial articulating surface 36 that is sized and oriented to articulate with the generally convex femoral articulating surface 20 during movement of the knee joint. Specifically, lateral compartment 56 of bearing portion 32 of tibial component 14 articulates with lateral condyle 18 of femoral component 12, and medial compartment 58 of bearing portion 32 of tibial component 14 articulates with medial condyle 19 of femoral component 12. To promote smooth articulation between tibial component 14 and femoral component 12, bearing portion 32 may be constructed of a polymer, including, but not limited to, a hydrogel, polyether ether ketone (PEEK), fiber reinforced polyether ether ketone, ultrahigh molecular weight polyethylene (UHMWPE), crosslinked ultrahigh molecular weight polyethylene, or polyether ketone ether ether ketone.

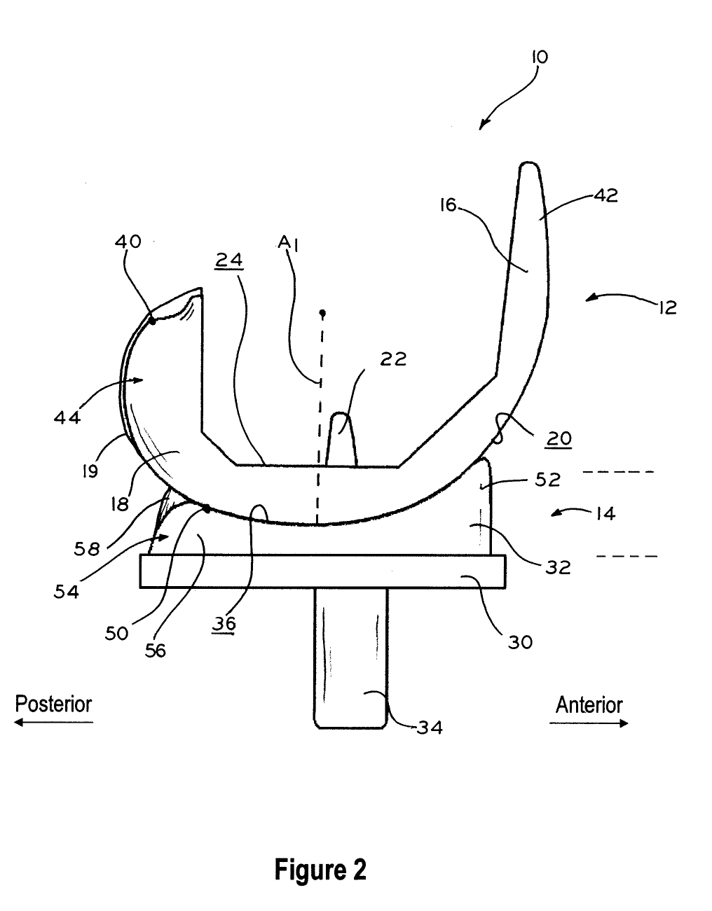

Referring next to FIG. 2, knee arthroplasty system 10 is illustrated in an orientation corresponding to an extended position of a leg. In this extended orientation, femoral component 12 rests upright against tibial component 14. If the patient were standing erect, a vertical plane would extend through both femoral component 12 and tibial component 14. As shown in FIG. 2, distal non-articulating surface 24 of femoral component 12 extends essentially parallel to, or approximately 0 degrees from, base 30 of tibial component 14.

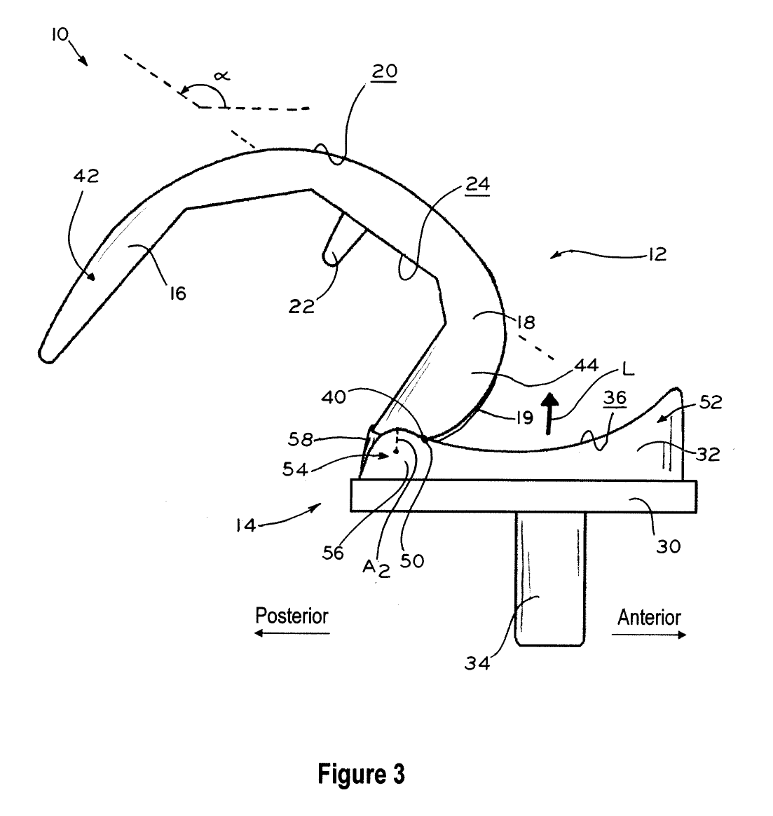

Referring from FIG. 2 to FIG. 3, as the patient bends or flexes the knee joint, such as to kneel or squat, femoral component 12 tilts posteriorly relative to tibial component 14 (FIG. 3). Additionally, the condyles of femoral component 12, including lateral condyle 18 and medial condyle 19, translate posteriorly across tibial component 14. In the flexed position of FIG. 3, relative to the extended position of FIG. 2, angle alpha (α) between distal non-articulating surface 24 of femoral component 12 and base 30 of tibial component 14 may exceed approximately 130 degrees, 140 degrees, 150 degrees, 160 degrees, or 170 degrees, for example. Moreover, angle (α) corresponds to a level of flexion in a knee with knee arthroplasty system 10 implanted therein, i.e., an angle between a tibial axis and a femoral axis. When femoral component 12 is articulated with tibial component 14, as described in detail below, knee arthroplasty system 10 moves between an extended position shown in FIG. 2 and flexed positions, one of which is shown in FIG. 3.

As used herein, a flexed position is a position in which knee arthroplasty system 10 is configured to correspond with flexion of a leg, such as for a kneeling or squatting motion. Conversely, an extended position corresponds to a standing position, while a hyperextended position corresponds to a knee extended past extension in the opposite direction of flexion.

To accommodate deep flexion of knee arthroplasty system 10, as shown in FIG. 3, femoral component 12, specifically lateral condyle 18 of femoral component 12, includes inflection point 40. At inflection point 40, the curvature of femoral articulating surface 20 as viewed in a sagittal plane changes from being convex to concave. For example, inflection point 40 is illustrated in FIGS. 1-3 in a sagittal plane intersecting lateral condyle 18 of femoral component 12, such as bisecting lateral condyle 18. However, any sagittal plane intersecting the concave portion of posterior end 44 of lateral condyle 18 would show inflection point 40. According to an exemplary embodiment of the present invention, inflection point 40 is located posteriorly on lateral condyle 18 of femoral component 12, so that inflection point 40 is proximate posterior end 44 of femoral articulating surface 20. Thus, anterior end 42 of femoral articulating surface 20 of lateral condyle 18 is generally convex and at least a portion of posterior end 44 of femoral articulating surface 20 of lateral condyle 18 is generally concave, while femoral articulating surface 20 of medial condyle 19 is substantially entirely convex. As shown in FIG. 3, femoral articulating surface 20 of lateral condyle 18 is mostly convex, with the convex anterior end 42 being longer or wider in the sagittal plane than the concave posterior end 44. Also, as shown in FIG. 3, the convex anterior end 42 has a larger radius of curvature than the concave posterior end 44.

Bearing portion 32 of tibial component 14, specifically lateral compartment 56 of bearing portion 32 formed on tibial component 14, may also include inflection point 50. At inflection point 50, the curvature of tibial articulating surface 36 as viewed in a sagittal plane changes from being concave to convex. For example, inflection point 50 is illustrated in FIGS. 1-3 in a sagittal plane intersecting lateral compartment 56 of tibial component 14, such as bisecting lateral compartment 56. However, any sagittal plane intersecting the convex portion of posterior end 54 of tibial articulating surface would show inflection point 40. According to an exemplary embodiment of the present invention, inflection point 50 is located posteriorly on lateral compartment 56 of bearing portion 32, so that inflection point 50 is proximate posterior end 54 of tibial articulating surface 36. Thus, in lateral compartment 56 of bearing portion 32, anterior end 52 of tibial articulating surface 36 is generally concave and posterior end 54 of tibial articulating surface 36 is generally convex. In this embodiment, bearing portion 32 of tibial component 14 may be asymmetric, both anteriorly-posteriorly and medially-laterally. As shown in FIG. 3, tibial articulating surface 36 is mostly concave, with the concave anterior end 52 being longer or wider in the sagittal plane than the convex posterior end 54. Also, as shown in FIG. 3, the concave anterior end 52 has a larger radius of curvature than the convex posterior end 54.

The posterior locations and smaller radii of concave posterior end 44 and convex posterior end 54 of femoral articulating surface 20 and tibial articulation surface 36, respectively, facilitate engagement of posterior ends 44, 54 at a flexion orientation of knee arthroplasty system 10, i.e. an orientation corresponding to relatively high degree of leg flexion. For example, a flexion orientation of knee arthroplasty system 10 may be in a flexed position in which angle alpha (α) (FIG. 3) exceeds at least about 130 degrees (as discussed above). However, it is within the scope of the present disclosure that posterior ends 44, 54 may occupy larger portions of their respective articulating surfaces 20, 36 by locating inflection points 40, 50 closer to anterior ends 42, 52. This anterior shift of inflection points 40, 50 would result in engagement of concave posterior end 44 and convex posterior end 54 at a lesser degree of flexion. Conversely, a posterior shift of inflection points 40, 50 would result in engagement of concave posterior end 44 and convex posterior end 54 at a higher degree of flexion.

According to an exemplary embodiment of the present invention, as femoral component 12 begins to flex relative to tibial component 14, the substantially convex femoral articulating surface 20 of femoral component 12 cooperates with the substantially concave tibial articulating surface 36 of tibial component 14, as shown in FIG. 2. Then, as knee arthroplasty system 10 reaches the more highly flexed position of FIG. 3, inflection point 40 of femoral component 12 corresponds with inflection point 50 of tibial component 14, such that the generally concave posterior end 44 of femoral articulating surface 20 cooperates with the generally convex posterior end 54 of tibial articulating surface 36. Femoral component 12 initially pivots about a larger radius of curvature relative to tibial component 14, illustrated schematically as radius A₁ in FIG. 2, and then femoral component 12 pivots about a smaller radius of curvature relative to tibial component 14, illustrated schematically as radius A₂ in FIG. 3. In the illustrated embodiment of FIG. 2, radius A₁ is either substantially equal in femoral component 12 and tibial component 14, such as in the early stages of flexion, or is smaller in femoral component 12 than in tibial component 14. Thus, femoral articulating surface 20 may highly conform to tibial articulating surface 36, or may have less conformity as required or desired for a particular application. Similarly, radius A₂ (FIG. 3) is generally equal in both femoral component 12 and tibial component 14, but may optionally be larger in femoral component 12 than in tibial component 14.

In the flexed position of FIG. 3, the interaction between the generally concave posterior end 44 of femoral articulating surface 20 and the generally convex posterior end 54 of tibial articulating surface 36 may provide posterior constraint to knee arthroplasty system 10, preventing femoral component 12 from dislocating posteriorly from tibial component 14 and thereby enhancing the overall stability of knee arthroplasty system 10 in deep flexion. Also, the generally concave posterior end 44 of femoral articulating surface 20 may cooperate with the generally convex posterior end 54 of tibial articulating surface 36 to lift femoral component 12 proximally away from tibial component 14, illustrated schematically as arrow L in FIG. 3. The continued posterior migration of femoral component 12 over tibial component 14 and the proximal lift-off of femoral component 12 from tibial component 14 may accommodate deep flexion of knee arthroplasty system 10 and may allow tibial component 14 to rotate relative to femoral component 12, similar to the behavior of a natural knee joint.

According to an exemplary embodiment of the present invention, lateral condyle 18 of femoral component 12 may have a larger radius of curvature than medial condyle 19 of femoral component 12. An exemplary femoral component is described in U.S. Pat. No. 6,770,099, filed Nov. 19, 2002, titled FEMORAL PROSTHESIS, and assigned to the assignee of the present application, the entire disclosure of which is expressly incorporated by reference herein. During flexion and extension, the larger lateral condyle 18 of femoral component 12 travels a greater distance over tibial component 14 than the smaller medial condyle 19 of femoral component 12, which may be described as “big wheel/little wheel” movement. Providing inflection point 40 on lateral condyle 18 of femoral component 12 and corresponding inflection point 50 in lateral compartment 56 of tibial component 14 may allow lateral condyle 18 of femoral component 12 to travel posteriorly relative to tibial component 14 until knee arthroplasty system 10 reaches deep flexion, similar to the behavior of a natural knee joint.

Referring from FIG. 3 back to FIG. 2, as the knee joint returns from the flexed position to the extended position, the generally concave posterior end 44 of femoral articulating surface 20 may cooperate with the generally convex posterior end 54 of tibial articulating surface 36 to tilt femoral component 12 anteriorly relative to tibial component 14. When inflection point 40 of femoral component 12 moves beyond or disengages from inflection point 50 of tibial component 14, the generally convex distal portion of femoral articulating surface 20 cooperates with the generally concave anterior end 52 of tibial articulating surface 36 to continue tilting femoral component 12 anteriorly relative to tibial component 14.

While this invention has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this invention pertains and which fall within the limits of the appended claims.

What is claimed is:

1. A prosthetic knee comprising:
a femoral component having an anterior femoral end and a posterior femoral end, said femoral component configured for securement to a resected distal femur, said femoral component comprising a femoral articulating surface extending in a sagittal plane between said anterior femoral end and said posterior femoral end, said femoral articulating surface having a femoral inflection point in the sagittal plane, said femoral articulating surface transitioning in the sagittal plane from a convex curvature to a concave curvature at said femoral inflection point; and
a tibial component having an anterior tibial end and a posterior tibial end, said tibial component configured for securement to a resected proximal tibia, said tibial component comprising a tibial articulating surface extending between said anterior tibial end and said posterior tibial end, said tibial articulating surface configured to articulate with said femoral articulating surface, said tibial articulating surface contacting said femoral inflection point at an angle of flexion of the prosthetic knee.

2. The prosthetic knee of claim 1, wherein said angle of flexion of the prosthetic knee at which said tibial articulating surface contacts said femoral inflection point equals at least 130 degrees of flexion of the prosthetic knee.

3. The prosthetic knee of claim 1, wherein said femoral inflection point is located proximate said posterior femoral end.

4. The prosthetic knee of claim 1, wherein said at least one condyle comprises a lateral condyle, and said femoral component further comprises a medial condyle, said femoral inflection point being located on said lateral condyle of said femoral component.

5. The prosthetic knee of claim 1, wherein said concave curvature of said femoral articulating surface defines a concave femoral radius, said convex curvature of said femoral articulating surface defines a convex femoral radius, said concave femoral radius smaller than said convex femoral radius.

6. The prosthetic knee of claim 1, wherein said tibial articulating surface comprises a tibial inflection point, said tibial articulating surface transitioning from a concave curvature to a convex curvature at said tibial inflection point.

7. The prosthetic knee of claim 6, wherein said concave curvature of said tibial articulating surface defines a concave tibial radius, said convex curvature of said tibial articulating surface defines a convex tibial radius, said concave tibial radius larger than said convex tibial radius.

8. The prosthetic knee of claim 6, wherein said tibial inflection point is located proximate said posterior tibial end.

9. The prosthetic knee of claim 6, wherein said tibial component comprises a bearing surface having a lateral tibial compartment and a medial tibial compartment, said tibial inflection point formed in said lateral tibial compartment.

10. The prosthetic knee of claim 9, wherein said femoral inflection point cooperates with said tibial inflection point to mate said concave curvature of said femoral component with said convex curvature of said tibial component when said prosthetic knee is in a flexion orientation.

11. The prosthetic knee of claim 10, wherein said femoral component includes a distal non-articulating surface and said tibial component includes a base surface, said distal non-articulating surface and said base surface substantially parallel when the prosthetic knee is in an extension orientation, said flexion orientation of the prosthetic knee corresponding to an angle between said distal non-articulating surface and said base surface exceeds about 130 degrees.

12. A prosthetic knee comprising:
a femoral component having an anterior femoral end and a posterior femoral end, said femoral component configured for securement to a resected distal femur, said femoral component comprising:
a medial condyle comprising a medial femoral articulating surface extending between said anterior femoral end and said posterior femoral end, said medial femoral articulating surface having a convex medial condyle curvature;
a lateral condyle comprising a lateral femoral articulating surface extending between said anterior femoral end and said posterior femoral end, said lateral femoral articulating surface having a substantially convex lateral condyle curvature; and
a femoral inflection point disposed on said lateral femoral articulating surface adjacent said posterior femoral end, said lateral femoral articulating surface transitioning from said substantially convex lateral condyle curvature to a concave lateral condyle curvature at said femoral inflection point, said concave lateral condyle curvature having a concave femoral radius; and
a tibial component having an anterior tibial end and a posterior tibial end, said tibial component configured for securement to a resected proximal tibia, said tibial component comprising:
a medial tibial compartment having a medial articulating surface extending between said anterior tibial end and said posterior tibial end, said medial tibial articulating surface sized and positioned to articulate with said medial femoral articulating surface of said medial condyle; and
a lateral tibial compartment having a lateral articulating surface extending between said anterior tibial end and said posterior tibial end, said lateral tibial articulating surface sized and positioned to articulate with said lateral femoral articulating surface of said lateral condyle; and
a tibial inflection point disposed on said lateral tibial articulating surface adjacent said posterior tibial end, said lateral tibial articulating surface transitioning from a concave tibial curvature to a convex tibial curvature at said tibial inflection point, said convex tibial curvature defining a convex tibial radius, said concave femoral radius at least as great as said convex tibial radius.

13. The prosthetic knee of claim 12, wherein said femoral inflection point cooperates with said tibial inflection point to mate said concave curvature of said femoral component with said convex curvature of said tibial component when said prosthetic knee is in a flexion orientation.

14. The prosthetic knee of claim 12, wherein:
said concave tibial curvature defines a concave tibial radius, said concave tibial radius larger than said convex tibial radius; and
said convex curvature of said femoral articulating surface defines a convex femoral radius, said concave femoral radius smaller than said convex femoral radius.

15. The prosthetic knee of claim 12, wherein said femoral component includes a distal non-articulating surface and said tibial component includes a base surface, said distal non-articulating surface and said base surface substantially parallel when the prosthetic knee is in an extension orientation, said flexion orientation of the prosthetic knee corresponding to an angle between said distal non-articulating surface and said base surface exceeds about 130 degrees.

16. A method of stabilizing a prosthetic knee in a configuration corresponding to deep flexion of a knee, the method comprising:
providing a femoral component having a femoral articulating surface with a convex femoral curvature and a concave femoral curvature, the convex femoral curvature transitioning to the concave femoral curvature at a femoral inflection point;
providing a tibial component having a tibial articulating surface with a concave tibial curvature and a convex tibial curvature, the convex tibial curvature transitioning to the concave tibial curvature at a tibial inflection point; and
articulating the femoral component with respect to the tibial component from an extension orientation to a flexion orientation, the convex femoral curvature engaging the concave tibial curvature in the extension orientation, and the concave femoral curvature engaging the convex tibial curvature in the flexion orientation.

17. The method of claim 16, wherein the flexion orientation corresponds to an angle between a distal non-articulating surface of the femoral component and a base surface of the tibial component of at least about 130 degrees.

18. The method of claim 16, wherein said step of providing a femoral component comprises forming the convex femoral curvature at an anterior femoral end and forming the concave femoral curvature at a posterior femoral end.

19. The method of claim 16, wherein said step of providing a femoral component comprises forming the concave femoral curvature with a smaller radius than the convex femoral curvature.

20. The method of claim 16, wherein said step of providing a tibial component comprises forming the concave tibial curvature at an anterior tibial end and forming the convex tibial curvature at a posterior tibial end.

21. The method of claim 16, wherein said step of providing a tibial component comprises forming the concave tibial curvature with a larger radius than the convex tibial curvature.
